(19) United States
(12) Patent Application Publication
(10) Pub. No.: US 2009/0247883 A1
(43) Pub. Date: Oct. 1, 2009

(54) BIOLOGICAL-BODY-ATTACHED DATA COMMUNICATION DEVICE

(76) Inventors: Shinichi Miyazaki, Osaka (JP); Hiroshi Kamiya, Osaka (JP); Yoshihisa Ohmoto, Osaka (JP); Osamu Matsui, Osaka (JP)

Correspondence Address: NEC CORPORATION OF AMERICA, 6535 N. STATE HWY 161, IRVING, TX, 75039, US

(21) Appl. No.: 12/413,306

(22) Filed: Mar. 27, 2009

(30) Foreign Application Priority Data
Mar. 31, 2008 (JP) 2008-094385

(51) Int. Cl. A61B 5/0205; A61B 5/02; A61B 5/01

(52) U.S. Cl. 600/483; 600/508; 600/549

(57) ABSTRACT

A biological-body-attached data communication device has a communication unit communicating with an external device via a biological body as a medium, a data processing unit transmitting/receiving data with the external device via the communication unit, an attachment detection unit like a heartbeat sensor detecting whether or not the biological-body-attached data communication device is attached to the biological body, and a power source unit. When the attachment detection unit detects that the device is attached to the biological body, the power source unit supplies operational power to the communication unit and the data processing unit, and when the attachment detection unit detects that the device is not attached to the biological body, the power source units terminates or suppresses power supply to those units. Accordingly, power consumption is suppressed, and draining of the power source is prevented.

### INCORPORATION BY REFERENCE

This application is based on Japanese Patent Application No. 2008-094385 filed on Mar. 31, 2008, and including specification, claims, drawings and summary. The disclosure of the above Japanese Patent Application is incorporated herein by reference in its entirety.

### TECHNICAL FIELD

The present invention relates to a biological-body-attached data communication device which is attached to a biological body and which performs communication through the biological body as a communication medium.

### BACKGROUND ART

There are known biological-body-attached data communication devices which perform data communication through a biological body as a communication medium. For example, Unexamined Japanese Patent Application KOKAI Publication No. 2002-259569 discloses a biological-body-attached data communication device that transmits a signal through a user's body when the user uses a toilet.

Conventional biological-body-attached data communication devices have a battery as a power source from the standpoint of the characteristic that such data communication devices are attached to a biological body, collect and process data using electrical energy supplied from the battery, and perform data communication.

In such biological-body-attached data communication devices, when the battery is drained, the devices are turned off, resulting in data loss, and the battery needs to be replaced. Accordingly, it is desired to extend the battery life and to suppress the number of battery replacement as few as possible. The capacity of the battery can be increased in order to extend the battery life, but the size and weight of the battery increase, so that the biological-body-attached data communication device itself becomes large, and it is not desirable from the standpoint of the characteristic that such a device is attached to a biological body.

Accordingly, a technology which can suppress the battery capacity but extend the battery life is demanded.

On the other hand, there is proposed a technology which determines a use environment and terminates unnecessary power supply in accordance with the use environment for the purpose of energy conservation. For example, Unexamined Japanese Patent Application KOKAI Publication No. H4-9140 discloses a snooze detection device which detects that a user dozes off, and automatically cuts off unnecessary power when the user is in a snoozing condition.

However, such a snooze detection device merely turns ON/OFF an external device, and such a publication does not disclose or suggest a technology which can suppress any power consumption of the snooze detection device itself operated by a battery. Accordingly, it is difficult to use this technology for a biological-body-attached data communication device to conserve energy, and is not effective.

### SUMMARY

The present invention has been made in view of the foregoing circumstances, and it is an exemplary object of the present invention to extend the battery life of a biological-body-attached data communication device.

To achieve the exemplary object, a biological-body-attached data communication device of the present invention is a biological-body-attached data communication device which is attached to a biological body and which communicates with an external device via the biological body as a communication medium, the device including:

a communication unit that performs communication via the biological body as a medium;

a data processing unit that performs data communication with the external device via the communication unit;

an attachment detection unit that detects whether or not the data communication device is attached to the biological body; and

a power source unit, and wherein

when the attachment detection unit is detecting that the data communication device is attached to the biological body, the power source unit supplies operational power to the communication unit, the data processing unit, and the attachment detection unit, and when the attachment detection unit is detecting that the data communication device is not attached to the biological body, the power source unit terminates or suppresses power supply to at least the communication unit.

A biological-body-attached data communication device of the present invention is a biological-body-attached data communication device which is attached to a biological body and which communicates with an external device via the biological body as a communication medium, the device including:

a communication means for performing communication via the biological body as a medium;

a data processing means for performing performs data communication with the external device via the communication means;

an attachment detection means for detecting whether or not the data communication device is attached to the biological body; and

a power source means, and wherein

when the attachment detection means is detecting that the data communication device is attached to the biological body, the power source means supplies operational power to the communication means, the data processing means, and the attachment detection means, and when the attachment detection means is detecting that the data communication device is not attached to the biological body, the power source means terminates or suppresses power supply to at least the communication means.

### BRIEF DESCRIPTION OF THE DRAWINGS

The object and other objects and advantages of the present invention will become more apparent upon reading of the following detailed description and the accompanying drawings in which:

FIG. 1 is a diagram showing the structure of a biological-body-attached data communication device according to the first embodiment of the present invention;

FIG. 2 is a diagram for explaining the operation of the biological-body-attached data communication device in FIG. 1;

FIG. 3 is a diagram showing the structure of a human-body-attached tag according to the second embodiment of the present invention;

FIG. 4 is a diagram for explaining how the power mode of the human-body-attached tag in FIG. 3 changes;

FIG. 5 is a diagram showing a configuration example of a heartbeat sensor;

FIG. 6 is a diagram showing the structure of a human-body-attached tag according to another embodiment of the present invention;

FIG. 7 is a diagram showing the stricture of a human-body-attached tag according to the other embodiment of the present invention; and

FIG. 8 is a diagram showing a configuration example of a computer which communicates with a human-body-attached tag of the present invention.

### EXEMPLARY EMBODIMENT

### First Embodiment

An explanation will be given of a biological-body-attached data communication device according to the first embodiment of the present invention with reference to the accompanying drawings.

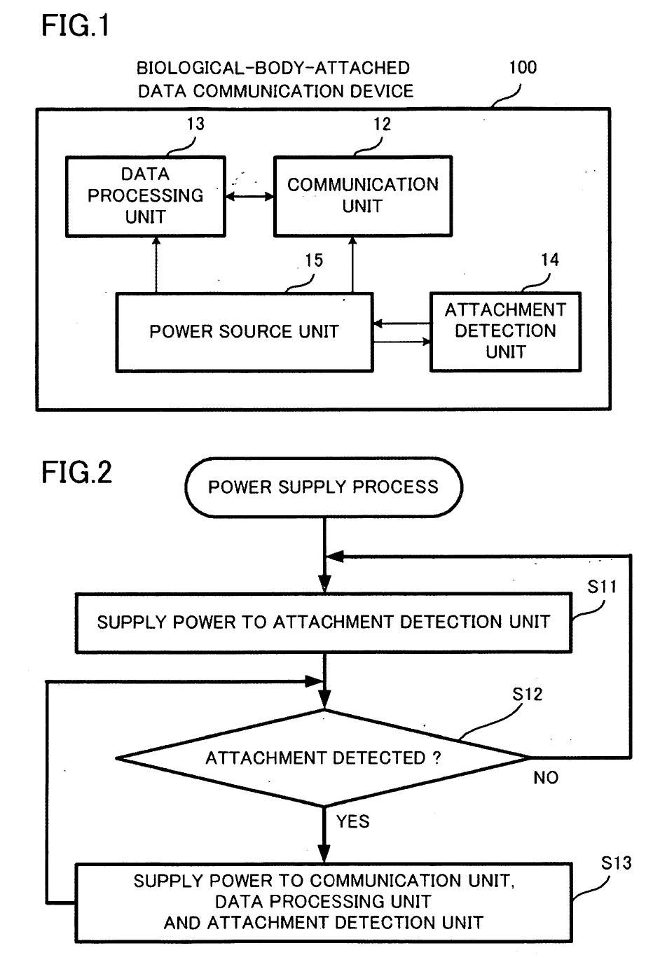

A biological-body-attached data communication device 100 of the embodiment is attached to a biological body like a human body, and communicates with an external device through the biological body as a communication medium. As shown in FIG. 1, the biological-body-attached data communication device 100 has a communication unit 12, a data processing unit 13, an attachment detection unit 14, and a power source unit 15.

The communication unit 12 has a transmission/reception electrode electrically connected to a biological body via an insulator, and performs data communication with an external device, e.g., a communication device provided at a toilet through the biological body as a communication medium.

The communication unit 12 modulates a carrier wave with, for example, a baseband signal subjected to transmission supplied from the data processing unit 13 to create a transmission signal, and transmits the created transmission signal to the external device through the transmission/reception electrode via the biological body as a communication medium. Moreover, for example, the communication unit 12 receives a signal transmitted from the external device via the biological body through the transmission/reception electrode, demodulates the received signal to reproduce a baseband signal, and supplies the reproduced baseband signal to the data processing unit 13.

The data processing unit 13 has a processor, an internal memory, and the like, and performs various kinds of data processing. For example, the data processing unit 13 collects various kinds of data from a non-illustrated sensor etc., processes the collected data to create data to be transmitted to the external device, and supplies such data to the communication unit 12. Moreover, as reception data from the external device is supplied from the communication unit 12, the data processing unit 13 processes such data.

The attachment detection unit 14 detects that the biological-body-attached data communication device 100 is attached to the biological body, i.e., the biological-body-attached data communication device 100 is in a significant condition, and supplies a detection signal to the power source unit 15. The attachment detection unit 14 comprises, for example, a heartbeat sensor, and outputs the detection signal when a heartbeat is detected.

The power source unit 15 has a battery, and supplies power stored in the battery to the foregoing individual units. When the biological-body-attached data communication device 100 is attached to the biological body, i.e., when the attachment detection unit 14 is outputting a detection signal, the power source unit 15 supplies power to all of the communication unit 12, the data processing unit 13, and the attachment detection unit 14. Conversely, when no detection signal is being output, the power source unit 15 supplies power only to the attachment detection unit 14, and terminates power supply to the communication unit 12 and the data processing unit 13.

Next, an explanation will be given of the operation of the biological-body-attached data communication device 100 having the foregoing structure with reference to the flowchart of FIG. 2.

When the operation starts, first, the power source unit 15 supplies operational power to the attachment detection unit 14, but does not supply power to the communication unit 12 and the data processing unit 13 (step S11). When the operational power supply to the attachment detection unit 14 is started, the attachment detection unit 14 starts operation, and starts detecting whether or not the biological-body-attached data communication device 100 is attached to the biological body. Initially, the attachment detection unit 14 detects that the device is not attached, and maintains a state of outputting no detection signal. During this state, the communication unit 12 and the data processing unit 13 do not operate so that no power is consumed.

Next, the power source unit 15 determines whether or not the attachment detection unit 14 outputs a detection signal, i.e., whether or not the biological-body-attached data communication device 100 is attached (step S12).

When the biological-body-attached data communication device 100 is attached to the biological body, the attachment detection unit 14 detects it, and outputs the detection signal. In response to the detection signal, the determination of the power source unit 15 becomes YES in the step S12, the power source unit 15 starts supplying power to the communication unit 12 and the data processing unit 13, and keeps supplying power to the attachment detection unit 14 (step S13).

Accordingly, the communication unit 12 and the data processing unit 13 are activated. Thereafter, the data processing unit 13 executes data processing. For example, the data processing unit 13 collects data, e.g., biological information, from a non-illustrated sensor or the like, and stores the data in the internal memory.

On the other hand, the communication unit 12 intermittently determines whether or not communication with the external device is possible through the transmission/reception electrode and the biological body.

For example, when the biological body to which the biological-body-attached data communication device 100 is attached, directly contacts the external device having a communication function, or the user has a wash using a toilet bowl having a communication function with the external device, the external device and the communication unit 12 are electrically connected together. Then, the communication unit 12 and the external device respectively determine that communication between those device and unit becomes possible.

When the communication with the external device becomes possible, the communication unit 12 sends a notification to that effect to the data processing unit 13.

In response to the notification, the data processing unit 13 supplies data stored in the internal memory to the external device through the communication unit 12 via the biological body as a communication medium. Moreover, the data processing unit 13 receives data transmitted from the external device through the communication unit 12, and stores such data in the internal memory.

While the biological-body-attached data communication device 100 is being attached to the biological body, the power source unit 15 repeats the step S12 and the step S13.

When the biological-body-attached data communication device 100 is removed from the biological body, the attachment detection unit 14 stops transmitting the detection signal, the flow in the step S12 becomes NO and goes to the step S11, and the power source unit 15 terminates power supply to the communication unit 12 and the data processing unit 13, but keeps supplying power to the attachment detection unit 14.

According to such a structure, before the biological-body-attached data communication device 100 is attached to the biological body, power from the power source unit 15 is consumed only by the attachment detection unit 14, thereby suppressing any battery consumption of the power source unit 15. Accordingly, it is possible to extend the battery life, and suppress the number of battery replacement. Moreover, as the biological-body-attached data communication device 100 is attached to the biological body, power from the power source unit 15 is supplied to the communication unit 12, the data processing unit 13, and the attachment detection unit 14, so that data processing and communication can be normally executed.

Note that the attachment detection unit 14 may be a type which does not require external power supply. This results in termination of power supply to the attachment detection unit 14 from the power source unit 15, thereby further suppressing power consumption.

### Second Embodiment

The second embodiment of the present invention embodies the biological-body-attached data communication device as a human-body-attached tag 200.

The human-body-attached tag 200 is a tiny portable device attached to a human body, has a heartbeat sensor embedded thereinside, stores detection data (heart rate of a user) from the heartbeat sensor, and transmits the stored data to an external device through the human body as a communication medium.

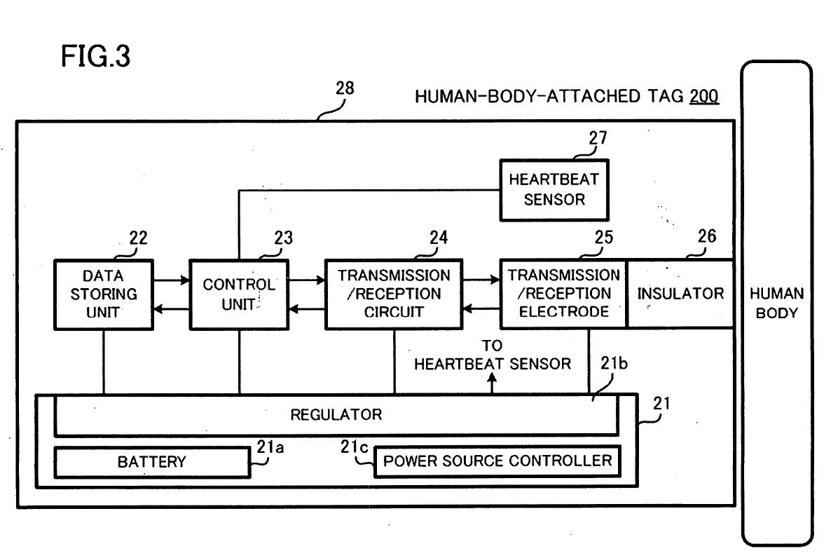

As shown in FIG. 3, the human-body-attached tag 200 has a power supply unit 21, a data storing unit 22, a control unit 23, a transmission/reception circuit 24, a transmission/reception electrode 25, an insulator 26, a heartbeat sensor 27, and a casing 28.

The power supply unit 21 has a battery 21a, a regulator 21b, a power source controller 21c, and the like embedded therein, causes the regulator 21b to convert power stored in the battery 21a into a voltage for the operation of each unit, and supplies such a voltage to each unit. The power source controller 21c comprises a power controlling processor or the like, and controls the power supply operation of the regulator 21b in response to a detection signal supplied from the heartbeat sensor 27.

The data storing unit 22 comprises a RAM (Random Access Memory), a ROM (Read Only Memory), and the like, and stores an operational program for the control unit 23 and control data therefor. The data storing unit 22 stores data indicating a heart rate measured by the heartbeat sensor 27.

The control unit 23 comprises a processor or the like, operates in accordance with the program stored in the data storing unit 22, and stores data indicating the heart rate detected by the heartbeat sensor 27 in the data storing unit 22 in association with, for example, the time at which the heart rate is detected, as heartbeat data. The control unit 23 encodes the heartbeat data temporarily stored in the data storing unit 22 to produce a baseband signal when transmitting data, and supplies the produced baseband signal to the transmission/reception circuit 24.

The control unit 23 receives data supplied from the external device via a human body through the transmission/reception electrode 25 and the transmission/reception circuit 24, and processes such data.

The transmission/reception circuit 24 modulates a carrier wave with the transmission-target baseband signal supplied from the control unit 23, amplifies the modulated signal, and supplies the amplified signal to the transmission/reception electrode 25. The transmission/reception circuit 24 receives a signal from the external device through the transmission/reception electrode 25, demodulates such a signal to produce a baseband signal, and supplies the produced baseband signal to the control unit 23.

The transmission/reception electrode 25 is electro-statically coupled (capacitive coupling) to a human body via the insulator (dielectric body) 26, transmits a signal (alternating-current signal) to the human body, and receives an alternating-current signal from the human body and supplies the received signal to the transmission/reception circuit 24.

The insulator 26 covers and insulates the transmission/reception electrode 25 so that the transmission/reception electrode 25 does not directly contact the human body (short-circuited), and electro-statically couples the transmission/reception electrode 25 to the human body.

The heartbeat sensor 27 is for detecting that the human-body-attached tag 200 is attached to the human body, and when detecting a heartbeat, supplies a detection signal and data indicating a heart rate to the control unit 23.

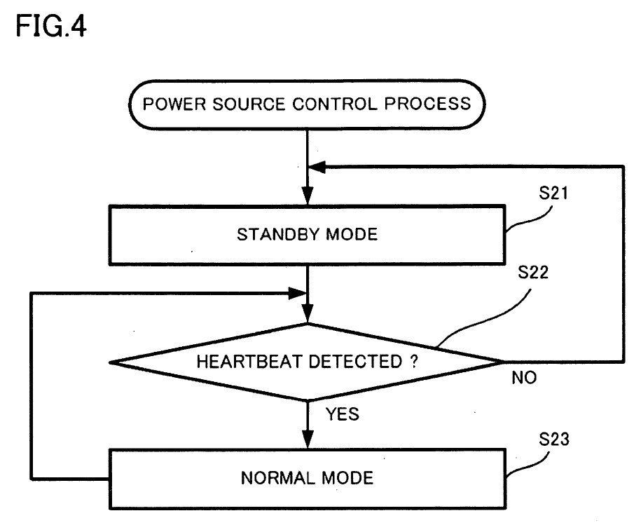

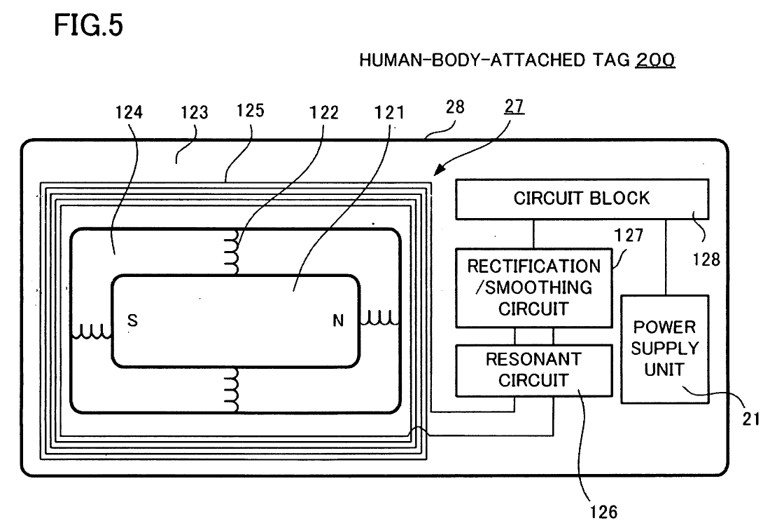

Next, an explanation will be given of the operation of the human-body-attached tag 200 having the foregoing structure with reference to the flowchart of FIG. 4.

In the initial state, the control unit 23 is in a standby mode (step S21), and instructs a standby mode to the power source controller 21c. In response to the instruction, tile power source controller 21c controls the regulator 21b to supply a low-voltage operation voltage VL for the standby mode (sleep mode) to the control unit 23, to apply a standby voltage which is merely sufficient for maintaining stored data (storing new data is difficult) to the data storing unit 22, and to apply a voltage for a normal operation which allows detection of a heartbeat to the heartbeat sensor 27. Note that no power is supplied to the transmission/reception circuit 24.

Next, the control unit 23 determines whether or not the heartbeat sensor 27 detects a heartbeat (step S22). Initially, since the human-body-attached tag 200 is not attached to a human body, the heartbeat sensor 27 detects no heartbeat. Accordingly, the determination in the step S22 becomes NO, and the power source controller 21c repeats the step S21 and the step S22.

When the human-body-attached tag 200 is attached to the human body, the heartbeat sensor 27 measures a heart rate, and supplies a detection signal and the counted number to the control unit 23. In response to the detection signal, the determination of the control unit 23 in the step S22 becomes YES, and the control unit 23 instructs the power source controller 21c to shift the mode from the standby mode to a normal mode (step S23).

In response to the instruction, the power source controller 21c controls the regulator 21b to supply a high-voltage (higher than VL) operation voltage VH for a normal operation to the control unit 23, to apply a normal operation voltage which allows storing of new data and updating of already-stored data to the data storing unit 22, to apply a voltage for a normal operation which allows detection of a heartbeat to the heartbeat sensor 27, and to supply an operation voltage which allows transmission reception of data to the transmission/reception circuit 24.

Afterward, the control unit 23 periodically stores a heart rate detected by the heartbeat sensor 27 as heartbeat data in the data storing unit 22. The control unit 23 stands by until a signal path for data transfer through the transmission/reception circuit 24 is established.

When a signal path between the human-body-attached tag 200 and the external device via the human body as a communication medium is established, the transmission/reception circuit 24 detects this, and notifies the control unit 23. The control unit 23 reads out heartbeat data stored in the data storing unit 22, encodes the heartbeat data to produce a baseband signal, and supplies the produced baseband signal to the transmission/reception circuit 24. The transmission/reception circuit 24 modulates a carrier wave with the supplied baseband signal to produce a transmission signal, amplifies the transmission signal and supplies the amplified signal to tile transmission/reception electrode 25.

The transmission/reception electrode 25 supplies the supplied transmission signal to the human body by electrostatic coupling, and transmits that signal to the destination external device via the human body as a medium.

The control unit 23 repeats the step S22 and the step S23 while the heartbeat sensor 27 is outputting a detection signal (i.e., while the human-body-attached tag 200 is being attached to the human body).

When the human-body-attached tag 200 is removed from the human body, the heartbeat sensor 27 becomes unable to detect a heartbeat, and terminates outputting of the detection signal. The control unit 23 detects this (step S22: NO), and shifts the mode to the standby mode (step S21).

As the mode is shifted to the standby mode, the control unit 23 instructs the power source controller 21c of the power supply unit 21 to change the mode to the standby mode. In response to the instruction, like the initial state, the power source controller 21c controls the regulator 21b to supply a low-voltage operation voltage VL for the standby mode to the control unit 23 to apply a standby voltage which is merely sufficient for maintaining stored data to the data storing unit 22, and to apply a voltage for a normal operation which allows detection of a heartbeat to the heartbeat sensor 27. The power source controller 21c terminates power supply to the transmission/reception circuit 24.

Afterward, the same power source control process is repeated.

As explained above, the human-body-attached tag 200 of the second embodiment monitors attachment to the human body through the heartbeat sensor 27, and performs power controlling depending on an attachment state to the human body. Accordingly, when the human-body-attached tag 200 is not attached to the user, power supply to individual units which do not need to operate is terminated, or, a low voltage/low power is supplied in order to suppress the requisite function as minimum as possible. Therefore, it is possible to reduce the power consumption of the human-body-attached tag 200, and to reduce the draining of the battery 21a.

In the second embodiment, the heartbeat sensor 27 may comprise, for example, as shown in FIG. 5, a thin-tabular permanent magnet 121, plural elastic members (e.g., springs) 122 supporting the permanent magnet 121, an antenna 125, a resonant circuit 126, and a rectification/smoothing circuit 127.

The permanent magnet 121 is supported in an opening 124 formed in a substrate 123 like a resin substrate or the like. The antenna 125 is formed around the opening 124 of the substrate 123 like a coil. The antenna 125 is connected to the resonant circuit 126 and the rectification/smoothing circuit 127. In a circuit block 128, units from the data storing unit 22 to the transmission/reception electrode 25 are formed, and an output signal from the rectification/smoothing circuit 127 is supplied to the circuit block 128 as a detection signal.

The weight of the permanent magnet 121 and the elastic constant, number, and layout of the elastic member 122 are adjusted and set appropriately. More specifically, a resonance frequency is set to 50 Hz to 80 Hz or so in such a way that the permanent magnet 121 resonates with vibration of a body surface due to heart beating of a human and vibration due to breathing when the human-body-attached tag 200 is attached to the chest of the human. Furthermore, the resonant circuit 126 is set to have a resonance frequency of 50 Hz to 80 Hz or so.

Next, an explanation will be given of the operation of the human-body-attached tag 200 having the heartbeat sensor 27 employing the forgoing structure.

When the human-body-attached tag 200 is not attached to the human body, the permanent magnet 121 does not vibrate, so that the output signal from the antenna 125 is almost 0 V. Accordingly, the output from the rectification/smoothing circuit 127 is also 0 V, and no detection signal is output.

Conversely, when the human-body-attached tag 200 is attached to the chest or the like of a human, the permanent magnet 121 resonates with vibration of a body surface due to a heartbeat or breathing, and the permanent magnet 121 vibrates in a relatively-large way Accordingly, a magnetic flux from the permanent magnet 121 traverses the antenna 125, and induced electromotive force is generated at the antenna 125. The frequency of the induced electromotive force is substantially equal to the resonant frequency of the resonant circuit 126, and the resonant circuit 126 amplifies a signal due to heart beating, attenuates a noise frequency, and outputs a signal. The rectification/smoothing circuit 127 rectifies and smoothes the alternating-current signal output from the resonant circuit 126, and supplies the signal to the control unit 23 in the circuit block 128.

According to the foregoing structure, the heartbeat sensor 27 can output a detection signal in accordance with attachment/detachment of the tag 200 without the need of power supply. Therefore, it is possible to further suppress power consumption when the tag 200 is not attached to the human body.

The present invention is not limited to the foregoing embodiments, and can be changed and modified in various forms.

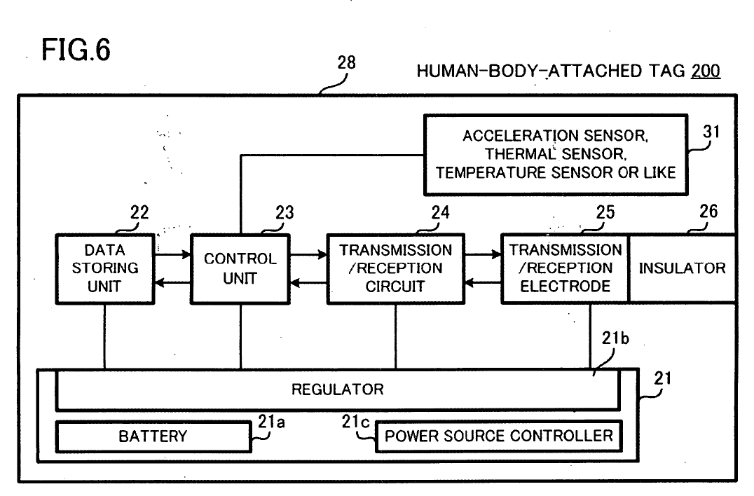

For example, in the foregoing embodiment, the heartbeat sensor 27 is used to detect the attached condition of the human-body-attached tag 200, but the other kind of a sensor can be used. For example, as shown in FIG. 6, an acceleration sensor 31 is disposed instead of the heartbeat sensor 27, and an acceleration when the tag is attached to a human body is detected, and power supply can be controlled in accordance with the detected acceleration. Moreover, a thermal sensor or a temperature sensor 31 is disposed instead of the heartbeat sensor 27, a heat from a human body or a body temperature is detected, and power supply can be controlled in accordance with the detection result.

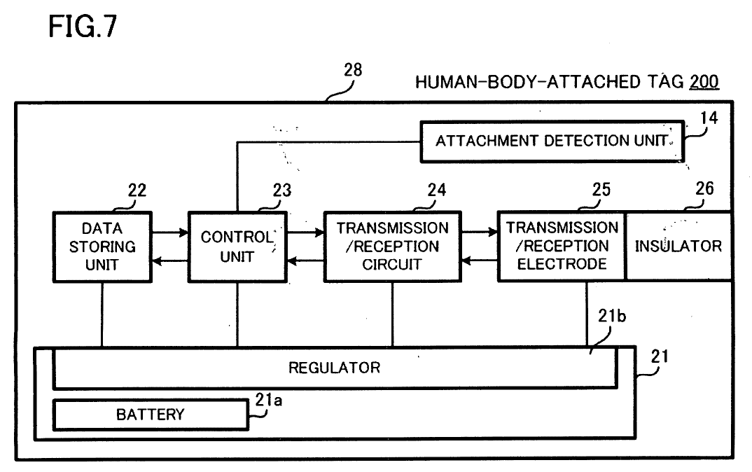

In the foregoing embodiment, the control unit 23 responds to a detection signal output from the attachment detection unit 14 like the heartbeat sensor 27, and instructs the power source controller 21c to control the power supply state. The present invention is, however, not limited to this case, and for example, as shown in FIG. 7, the power source controller 21c may be eliminated, and the control unit 23 may directly control the regulator 21b.

Moreover, a detection signal output from the attachment detection unit 14 may be supplied to the power source controller 21c, and the power source controller 21c may control the operation of the regulator 21b independently from the control unit 23. The procedures of the operation are the same as ones shown in FIGS. 2 and 4.

Next, an explanation will be given of usage examples of the human-body-attached tag 200 of the present invention.

### FIRST USAGE EXAMPLE

A user ID (Identification Information) is stored in the data storing unit 22 of the human-body-attached tag 200 beforehand.

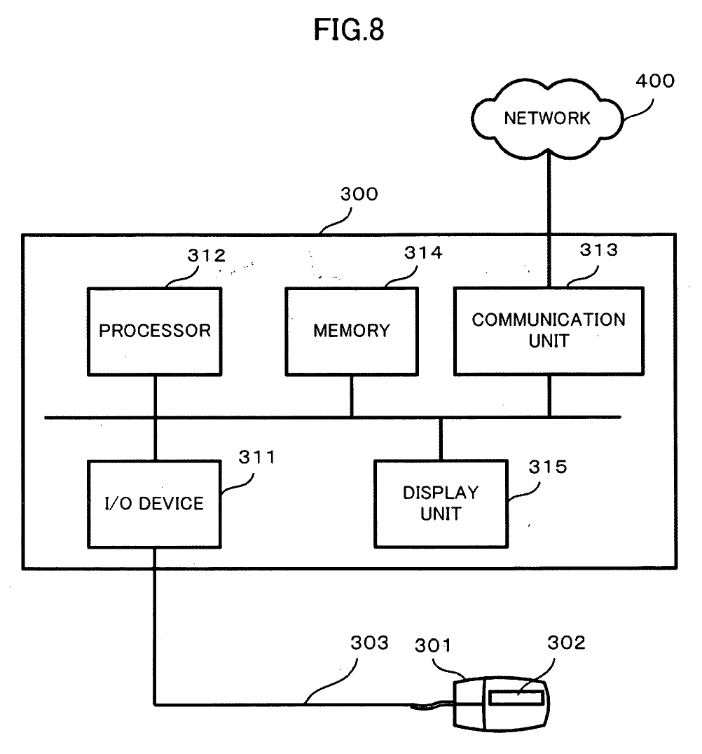

On the other hand, a mouse 301 connected to a computer 300 shown in FIG. 8 has an electrode 302 disposed thereon so that a user can unconsciously touch. The electrode 302 is connected to an I/O device (Input/Output device) 311 via a communication cable 303. The I/O device 311 has a function of performing serial communication with the transmission/reception circuit 24 of the human-body-attached tag 200. A processor 312 communicates with the human-body-attached tag 200 via the I/O device 311, and may communicate with a device over a network 400 via a communication unit 313.

When a user to whom the human-body-attached tag 200 is attached manipulates the mouse 301, the user unconsciously touches the electrode 302, and the transmission/reception circuit 24 of the human-body-attached tag 200 and the I/O device 311 of the computer 300 are connected together through the transmission/reception electrode 25, a human body, the electrode 302, and the cable 303.

Accordingly, communication between the processor 312 of the computer 300 and the control unit 23 of the human-body-attached tag 200 is enabled. Then, the control unit 23 transmits the user ID stored in the data storing unit 22 to the processor 312 through the transmission/reception circuit 24 and the transmission/reception electrode 25 via the human body as a medium. The processor 312 performs user authentication based on the user ID supplied from the human-body-attached tag 200. When the authentication is failed, the processor 312 does not perform a communication process with the human-body-attached tag 200 or ignores a process command or the like transmitted from the human-body-attached tag 200.

A memory 314 in the computer 300 stores a correspondence table between the user ID and data or a file that the user specified by the user ID can access. The correspondence table is stored in a memory area where an external access is prohibited.

When the authentication is succeeded, the processor 312 refers to the correspondence table with the user ID being as a key, and determines data or a file that is accessible. Thereafter, the processor 312 permits the user to access data or a file that the user has an access authority among data and files stored in the memory 314.

On the other hand, the human-body-attached tag 200 always collects the biological data of the user, such as a heart rate, an electrocardiogram, a body temperature, and a blood pressure, and stores such data in the data storing unit 22. When the human-body-attached tag 200 is connected to the computer 300, the human-body-attached tag 200 transmits such data and a process command to the computer 300. In response to the process command, the processor 312 processes the received biological data.

For example, the processor 312 stores the received biological data in the memory 314, acquires the history of the biological data, and displays history information on the display unit 315, and, when detecting any variation and abnormality from the history, displays an annunciation to that effect on the display unit 315. Or, the processor 312 provides such data to the server of an exclusive site (ASP (Application Service Provider) via the communication unit 313 and the network 400, receives diagnosis data from the server, and displays the diagnosis data on the display unit 315.

### SECOND USAGE EXAMPLE

In order to facilitate the user to use the computer 300, it is possible to change the setting of the computer 300 in accordance with the preference of the user when the user touches the computer 300. An explanation will be given of this case.

The data storing unit 22 of the human-body-attached tag 200 stores a user ID beforehand in association with setting information for the user.

When a user authentication successes, the processor 312 of the computer 300 reads out the setting information of itself (various setting parameters), and transmits such parameters to the control unit 23 of the human-body-attached tag 200. The control unit 23 saves the received setting information (setting information for restoring) in the data storing unit 22.

Next, the control unit 23 transmits setting information stored in the data storing unit 22, i.e., setting information matching the preference of the user, to the processor 312. In accordance with the notified setting information, the processor 312 reconfigures the computer 300. Afterward, the user can use the computer 300 in a setting condition usually used.

When the user finishes using the computer 300, the user instructs an “end” to the processor 312. In response to the instruction, the processor 312 requests the control unit 23 of tile human-body-attached tag 200 to transmit the setting information saved in the data storing unit 22. The control unit 23 responds to the request, reads out the setting information for restoring saved in the data storing unit 22, and transmits the setting information to the processor 312 of the computer 300. In accordance with the received setting information, the processor 312 reconfigures the computer 300. Accordingly, the computer 300 returns to the original setting condition.

### THIRD USAGE EXAMPLE

The result of a data processing or the like can be stored in the data storing unit 22 of the human-body-attached tag 200 when the data processing by the computer 300 is completed. When the user uses the computer 300 next time, the processor 312 may use the previous data stored in the data storing unit 22 of the human-body-attached tag 200 to do data processing. An explanation will be given of this case.

In this case, for example, the data storing unit 22 of the human-body-attached tag 200 stores a user ID and the result of a data processing by the computer 300 (data processing result information).

When the user to whom the human-body-attached tag 200 is attached touches the mouse 301, the user touches the electrode 302, so that the transmission/reception circuit 24 of the human-body-attached tag 200 and the computer 300 become possible to do data communication.

The control unit 23 transmits the user ID stored in the data storing unit 22 to the processor 312 of the computer 300. The processor 312 performs user authentication based on the transmitted user ID. When the user authentication successes, the processor 312 transmits the notification to that effect to the control unit 23 of the human-body-attached tag 200. In response to the notification, the control unit 23 transmits data processing result information stored in the data storing unit 22 to the processor 312. The processor 312 performs data processing based on the received data processing result information.

When a program reaches a predetermined check point or completes a predetermined process, the processor 312 transmits the result of the data processing to the control unit 23 of the human-body-attached tag 200. The control unit 23 stores the received result of the data processing as data processing result information in the data storing unit 22.

The content of the data processing is arbitrary. An example of such a result of the data processing is information on a play of a game (computer game).

In this case, the data storing unit 22 stores, for example, status (report) information, the progress of the game (e.g., to which stage the game has progressed), information for setting a scene of a game (e.g., various parameter information for a character, a point and item etc. acquired by a player) as data processing result information.

For example, When a player to whom the human-body-attached tag 200 is attached manipulates the mouse 301, the transmission/reception circuit 24 of the human-body-attached tag 200 and the computer 300 becomes possible to communicate with each other, and the control unit 23 of the human-body-attached tag 200 transmits a user ID stored in the data storing unit 22 to the processor 312 of the computer 300. The processor 312 performs user authentication based on the transmitted user ID. As the user authentication successes, the processor 312 transmits a notification to that effect to the control unit 23. The control unit 23 reads out status information from the data storing unit 22, and transmits such information to the processor 312.

The processor 312 proceeds with the game based on the received status information (in other words, the processor 312 reconstructs the scene of the game based on the received status information and then proceeds with the game).

When the game progresses and reaches a predetermined progress level, i.e., a predetermined save point, the processor 312 transmits game status information at that point to the control unit 23. The control unit 23 overwrites and saves the received status information in the data storing unit 22.

According to such a saving scheme, status information is automatically saved in the human-body-attached tag 200, and is automatically provided to the computer 300 from the human-body-attached tag 200 at the time of next play. Therefore, a user can enjoy the game without a bothersome saving operation.

Note that statuses of plural games can be stored in the data storing unit 22 of the human-body-attached tag 200. In this case, the processor 312 of the computer 300 transmits a game ID of a running game to the control unit 23 of the human-body-attached tag 200 when needed as the game progresses. The control unit 23 reads out status information corresponding to the transmitted game ID from the data storing unit 22, and supplies the status information to the processor 312. As the game progresses and reaches a predetermined progress level, i.e., a predetermined save point, the processor 312 transmits the game ID of the running game and the status information of the game to the control unit 23. The control unit 23 stores the received status information in the data storing unit 22 in association with the received game ID.

Moreover, in a game using a play medium (e.g., Japanese pinball), the human-body-attached tag 200 can be used as a medium storing the number of held play media.

In this case, for example, as money is deposited in a play medium renting device (computer 300), the processor 312 notifies data indicating the number of play media corresponding to the amount of the deposited money to the control unit 23 of the human-body-attached tag 200. The control unit 23 stores data indicating the notified number in the data storing unit 22. When the number of play media is already stored in the data storing unit 22, such number may be replaced with a total number that the additional number is added.

When a user plays a game using a game machine (computer 300), the control unit 23 of the human-body-attached tag 200 notifies the number of play media stored in the data storing unit 22 to the game machine. The processor 312 of the game machine permits the issuance (lending) of the play media up to the number of play media notified from the human-body-attached tag 200. When the game machine issues the play media, the processor 312 notifies the number of issued play media to the control unit 23, and the control unit 23 updates the number of play media stored in the data storing unit 22 in such a way that the number of play media is decreased by what corresponds to the issued number.

When the user instructs the game machine or a checkout machine (computer 300) to save the play media, the number of play media is counted, and the counted value is notified to the control unit 23. The control unit 23 adds the notified play media number to the play media number stored in the data storing unit 22.

### FOURTH USAGE EXAMPLE

When a data processing by the computer 300 is completed, data indicating the result of the data processing or the like may be stored in an arbitrary memory device over the network 400, and the address of the memory device and authentication information may be stored in the data storing unit 22 of the human-body-attached tag 200. In this case, when the user uses the computer 300 next time, the processor 312 acquires the address of the memory device over the network 400 and the authentication information from the human-body-attached tag 200. The processor 312 reads out data indicating the result of the data processing or the like from the memory device based on the acquired information, and continues the data processing using the read-out data.

According to such a structure, successive data processing becomes possible using an arbitrary computer 300.

Furthermore, the biological-body-attached data communication device can be used in other arbitrary situations.

The kind and format of data collected and transmitted by the biological-body-attached data communication device are optional. The present invention can be widely applied to not only a human body but also data transmission/reception device transmitting/receiving data via a biological body.

In the foregoing embodiment, the explanation has been given of the case where the transmission/reception electrode 25 contacts a human body via the insulator 26 by electrostatic (capacitance) coupling, but the transmission/reception electrode 25 may directly contact the human body, and the form of the electrode may be selected depending on the kind of a signal to be transmitted/received.

Various embodiments and changes may be made thereunto without departing from the broad spirit and scope of the invention. The above-described embodiments are intended to illustrate the present invention, not to limit the scope of the present invention. The scope of the present invention is shown by the attached claims rather than the embodiments. Various modifications made within the meaning of an equivalent of the claims of the invention and within the claims are to be regarded to be in the scope of the present invention.

What is claimed is:

1. A biological-body-attached data communication device which is attached to a biological body and which communicates with an external device via the biological body as a communication medium the device comprising:
a communication unit that performs communication via the biological body as a medium;
a data processing unit that performs data communication with the external device via the communication unit;
an attachment detection unit that detects whether or not the data communication device is attached to the biological body; and
a power source unit, and wherein
when the attachment detection unit is detecting that the data communication device is attached to the biological body, the power source unit supplies operational power to the communication unit, the data processing unit, and the attachment detection unit, and when the attachment detection unit is detecting that the data communication device is not attached to the biological body, the power source unit terminates or suppresses power supply to at least the communication unit.

2. The biological-body-attached data communication device according to claim 1, wherein:
the attachment detection unit comprises a heartbeat sensor; and
the power source unit supplies power to the data processing unit and the communication unit when the heartbeat sensor is detecting a heartbeat, and terminates or suppresses power supply to at least the communication unit when the heartbeat sensor does not detect a heartbeat.

3. The biological-body-attached data communication device according to claim 2, wherein the heartbeat sensor comprises:
a magnet;
an elastic member that supports the magnet in a manner that the magnet can resonates with a vibration of a body surface;
an antenna that generates induced electromotive force originating from the vibration of the magnet; and
a circuit that outputs the induced electromotive force of the antenna.

4. The biological-body-attached data communication device according to claim 1, wherein the attachment detection unit comprises an acceleration sensor that detects an acceleration when the data communication-device is attached to the biological body.

5. The biological-body-attached data communication device according to claim 1, wherein the attachment detection unit comprises a thermal sensor or a temperature sensor.

6. The biological-body-attached data communication device according to claim 1, further comprising a biological information collection unit that collects biological information of a person that attaches the data communication device, and wherein:
the power source unit terminates or suppresses power supply to the biological information collection unit when the attachment detection unit is detecting that the data communication device is not attached to the biological body; and
the communication unit transmits-data collected by the biological information collection unit to the external device.

7. The biological-body-attached data communication device according to claim 1, further comprising a memory unit that stores user setting information for setting a computer to be a predetermined setting condition, and authentication information, and wherein
when a communication with the computer which is the external device becomes possible, the data processing unit transmits the authentication information to the computer, when authentication successes, the data processing unit saves restore setting information transmitted from the computer and indicating the setting condition of the computer at this time in the memory unit, transmits the user setting information stored in the memory unit to the computer, and transmits the saved restore setting information to the computer in response to a notification from the computer to the effect that a process is completed.

8. The biological-body-attached data communication device according to claim 1, further comprising a memory unit that stores data processing result information which is a process result of a data processing by a computer, and authentication information, and wherein
when a communication with the computer which is the external device becomes possible, the data processing unit transmits the authentication information to the computer, when authentication successes, the data processing unit transmits the data processing result information stored in the memory unit to the computer, and when receiving a process result of a present data processing from the computer, stores the received process result of the present data processing in the memory unit as data processing result information.

9. A biological-body-attached data communication device which is attached to a biological body and which communicates with an external device via the biological body as a communication medium, the device comprising:
a communication means for performing communication via the biological body as a medium;
a data processing means for performing performs data communication with the external device via the communication means;
an attachment detection means for detecting whether or not the data communication device is attached to the biological body; and
a power source means, and wherein
when the attachment detection means is detecting that the data communication device is attached to the biological body, the power source means supplies operational power to the communication means, the data processing means, and the attachment detection means, and when the attachment detection means is detecting that the data communication device is not attached to the biological body, the power source means terminates or suppresses power supply to at least the communication means.
